(19) United States
(12) Patent Application Publication
(10) Pub. No.: US 2008/0145876 A1
(43) Pub. Date: Jun. 19, 2008

(54) POLY(ETHYLENE GLYCOL) ANTI-BODY DETECTION ASSAYS AND KITS FOR PERFORMING THEREOF

(75) Inventors: Jonathan K. Armstrong, Los Angeles, CA (US); Timothy C. Fisher, La Crescenta, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 11/943,532

(22) Filed: Nov. 20, 2007

Related U.S. Application Data
(60) Provisional application No. 60/866,756, filed on Nov. 21, 2006.

(51) Int. Cl. G01N 33/53; G01N 33/542; G01N 33/00; G01N 33/543

(52) U.S. Cl. 435/7.25; 435/7.1; 435/7.9; 436/518

(57) ABSTRACT

The present invention discloses an assay for determining the presence of an anti-PEG antibody in a biological sample. Embodiments according to this aspect of the present invention will generally have the steps of: (1) providing an antigen probe capable of forming an antibody-antigen complex with the anti-PEG antibody; (2) contacting the biological sample with the antigen probe under conditions favorable for formation of the antibody-antigen complex; and (3) analyzing the antigen probe, after having performed step (2), to detect for the presence of the antibody-antigen complex, wherein the presence of the anti-PEG antibody is determined if the antibody-antigen complex is detected. Also disclosed are methods for screening patients, methods for monitoring patients using assays of this invention and kits for performing thereof.

### CROSS-REFERENCE TO RELATED APPLICATIONS

This application claims an invention which was disclosed in Provisional Application Number 60/866,756 filed Nov. 21, 2006, entitled “POLY(ETHYLENE GLYCOL) ANTI-BODY DETECTION KIT”. The benefit under 35 USC §119(e) of the U.S. provisional application is hereby claimed. The above priority application is hereby incorporated herein by reference.

### STATEMENT OF FEDERALLY SPONSORED RESEARCH AND DEVELOPMENT

The present invention is made, at least in part, with the support of a grant from National Institute of Health, grant number HL65637. The government has certain rights in the invention.

### FIELD OF THE INVENTION

The present invention relates generally to assays for screening biological samples to determine the presence or absence of an antibody against poly(ethylene glycol). The present invention also relates to methods for using the assay to screen and monitor patients as well as kits and reagents for performing thereof

### BACKGROUND OF THE INVENTION

There are many hormones, enzymes and other proteins that are, or could be, very useful for the treatment of diseases, but which are also highly immunogenic. If administered to humans, such substances may elicit a severe hypersensitivity reaction and/or be cleared very rapidly from the body by the immune system. One generally accepted approach for circumventing these limiting effects is to covalently conjugate the drugs to poly(ethylene glycol) (PEG) so as to mask the drugs from the body's immune system. PEG is a nonionic, water soluble synthetic polymer that is widely used in the food, cosmetic and pharmaceutical industries. It was first introduced for commercial applications in the early 1950s. PEG is a nontoxic compound, and has been long believed to be non-immunogenic and non-antigenic. These properties are ideal for masking other substances with less than ideal immunogenic profiles. The development of PEG-conjugation has greatly extended the residence time in the circulation and greatly improved the efficacy of numerous therapeutically-useful proteins and enzymes. Some examples of PEG-conjugated therapeutic agents currently in clinical use include PEG-interferon, PEG-adenosine deaminase and PEG-asparaginase.

However, there is a growing body of evidence suggesting that PEG-conjugated therapeutics are ineffective for some patients due to a specific antibody directed against the PEG molecule (anti-PEG) which causes rapid clearance of the PEG-conjugate. Further, some patients may initially respond to a PEG-conjugated therapeutic, but later show a diminished or absent response due to subsequent development of an anti-PEG antibody. From a clinical standpoint, it would clearly be advantageous to be able to pre-screen a patient for the existence of anti-PEG before a PEG-conjugated therapeutic agent is administered, to establish whether it is likely to be well-tolerated and effective for that patient. For patients with evidence of anti-PEG and who are likely to have an inadequate response to a PEG-conjugated therapeutic, alternative treatment modalities (if available) may be more effective and lead to a better outcome.

Richter and Åkerblom described a passive hemagglutination test using poly(ethylene glycol)-conjugated RBCs (PEG-RBCs) with rabbit sera samples (1). Serial dilutions of sera were incubated with an equal volume of a 2% suspension of PEG-RBCs for 2 hours. The settling patterns of PEG-RBCs were observed and interpreted as a positive or negative result for the presence of anti-PEG. These measurements were expressed as reciprocals of the highest serum dilution yielding complete agglutination. The same test was later applied by Richter and Åkerblom to examine the sera of healthy human donors and allergy patients. Using this test, they identified anti-PEG in 1 of 500 healthy persons (0.2%) and in 3.3% of the allergic patients (2). From these results, Richter and Åkerblom concluded that anti-PEG had no clinical significance for PEG-modified allergens in hyposensitization therapy (2). The testing protocol described by Richter and Åkerblom is time consuming, requires a series dilution of patient sera, requires a subjective assessment of hemagglutination, did not use adequate controls to identify false positive or negative results, and most importantly, has very low sensitivity. Richter and Åkerblom found a prevalence of 0.2% in healthy donors; by comparison the anti-PEG assays described herein have consistently shown a prevalence of anti-PEG of about 25% in healthy normal subjects. i.e., Richter and Åkerblom's technique underestimates the prevalence by a factor of 100. Note that also in contrast to Richter and Åkerblom's data, the prevalence of 25% was confirmed by the use of appropriate positive and negative controls and antibody isotype analysis (see examples).

More recently, Ganson et al. (3) described an ELISA in which immobilized PEG-uricase was employed as an antigen to detect antibodies in serum samples from patients who had received PEG-uricase as a treatment for refractory gout. The assay included a 1 hour incubation at 37° C. followed by an overnight incubation at 4° C., and also washing steps employing Tween-20, a poly(ethylene glycol)-sorbitan monolaurate block copolymer surfactant. The authors suggested that a positive result in the assay indicated the presence of anti-PEG in the patient's serum. However, their assay design would not be able to distinguish between antibodies to PEG (anti-PEG) and antibodies directed against: a) the highly immunogenic uricase component of the conjugate (anti-uricase); or b) an antigenic epitope exposed by or created during the PEG-modification of the enzyme (anti-PEG-uricase). Further, the use of Triton (a PEG-containing molecule) in the washing buffer results in conditions that would not be expected to favor the formation of a detectable antigen:antibody complex in the assay. Hence, Ganson et al. does not teach an assay technique suitable for detecting an antibody specific to poly(ethylene glycol).

There is presently no assay available to rapidly screen and identify patients with anti-PEG. Therefore, there exists a great need for an assay that is capable of rapidly screening and identifying patients who are anti-PEG positive.

### SUMMARY OF THE INVENTION

Accordingly, in one aspect, the present invention provides an assay for determining the presence of an anti-PEG antibody in a biological sample. Embodiments according to this aspect of the present invention will generally have the steps of: (1) providing an antigen probe capable of forming an antibody-antigen complex with the anti-PEG antibody; (2) contacting the biological sample with the antigen probe under conditions favorable for formation of the antibody-antigen complex; and (3) analyzing the antigen probe, after having performed step (2), to detect for the presence of the antibody-antigen complex, wherein the presence of the anti-PEG antibody is established if the antibody-antigen complex is detected.

In another aspect, the present invention also provides a method for screening a patient prior to administering a PEG-conjugated therapeutic agent. Methods in accordance with this aspect of the present invention will generally have the steps of: (1) obtaining a biological sample from the patient; (2) contacting the biological sample with an antigen probe capable of forming an antigen-antibody complex with an anti-PEG antibody under conditions favorable for formation of the antigen-antibody complex; and (3) analyzing the antigen probe, after having performed step (2), to detect for the presence of the antigen-antibody complex, wherein if the antigen-antibody complex is detected, presence of anti-PEG in the patient's sample is confirmed, and therefore the patient may be expected to show a diminished response, or no response at all, to the PEG-conjugated therapeutic agent.

In yet another aspect, the present invention also provides a method for monitoring a patient's response to a PEG-conjugated therapeutic agent. Methods in accordance with this aspect of the present invention will generally have the steps of: (1) administering a PEG-conjugated therapeutic agent to a patient; (2) obtaining a biological sample from the patient; (3) contacting the biological sample with an antigen probe capable of forming an antigen-antibody complex with an anti-PEG antibody under conditions favorable for formation of the antigen-antibody complex; (4) analyzing the antigen probe after step (2) to detect for the presence of the antigen-antibody complex; and (5) repeating steps (2)-(4) at predetermined intervals, wherein when the antigen-antibody complex is detected, the presence of anti-PEG is confirmed, indicating that the patient has developed antibodies to PEG and may be expected to show a diminished response, or no response, to further doses of the PEG-conjugated therapeutic agent.

In still another aspect of the present invention, there is provided a kit for performing assays and methods as described in the above aspects of the present invention. In generally, a kit in accordance with embodiments of the present invention will have an antigen probe capable of forming an antigen-antibody complex with an anti-PEG antibody.

Other aspects and advantages of the invention will be apparent from the following description and the appended claims.

### BRIEF DESCRIPTION OF THE DRAWINGS

FIG. 1 shows the result of a serological test using PEG-coated RBCs. Sample details are 1P) Buffer (negative); 2P) weak positive (2+w); 3P) weak positive (1+−2+w), 4P) strong positive (4+); 5P) negative, plasma sample from 4P pre-incubated with TentaGel beads to remove anti-PEG, 6P) negative.

FIG. 2 shows the result of a gel test using PEG-coated RBCs. Sample details are Ctrl) anti-PEG negative control plasma samples, PEG) and PEG2) anti-PEG positive plasma samples.

FIG. 3 shows the result of an assay in accordance with embodiments of the present invention with flow cytometric outputs using 10 μm diameter spherical TentaGel-OH beads stained for bound immunoglobulins with fluorescent secondary antibodies.

FIG. 4 shows images of serology tube testing results for various PEG-RBC incubation conditions described in Table 1. Plasma samples (Sample 1=negative for anti-PEG, Samples 2 and 3=positive for anti-PEG).

FIG. 5 shows images of serology gel test results for various PEG-RBC incubation conditions described in Table 1. Plasma samples (Sample 1=negative for anti-PEG, Samples 2 and 3=positive for anti-PEG).

FIG. 6 shows serological identification of anti-PEG versus asparaginase activity for PEG-asparaginase treated ALL patients. Anti-PEG positive patients (squares, n=9) show undetectable ASNase activity while anti-PEG negative patients (triangles, n=19) show a mean ASNase activity of 353 U/L.

FIG. 7 shows cytometric identification of IgM anti-PEG. Anti-PEG positive patients (squares, n=13) show negligible ASNase activity (mean=12 U/L). Anti-PEG negative patients (triangles, n=15) show a high ASNase activity (mean=438 U/L).

Mean data are shown with open symbols±S.D.

### DETAILED DESCRIPTION

As set forth in the summary section above, the present invention has a number of different aspects, each having numerous possible embodiments.

In one aspect, the present invention provides an assay for determining the presence of an anti-PEG antibody in a biological sample. Embodiments according to this aspect of the present invention will generally have the steps of: (1) providing an antigen probe capable of forming an antibody-antigen complex with the anti-PEG antibody; (2) contacting the biological sample with the antigen probe under conditions favorable for formation of the antibody-antigen complex; and (3) analyzing the antigen probe, after having performed step (2), to detect for the presence of the antibody-antigen complex, wherein the presence of the anti-PEG antibody is established if the antibody-antigen complex is detected.

As used herein, the term “biological sample” refers to samples obtained from a test subject. In preferred embodiments, the biological sample is a bodily fluid samples containing antibodies. This can be blood serum, or plasma. In other embodiments, bodily fluids or other types of biological samples such as organ tissues or cell cultures may also be advantageously used depending on the specific assay analysis technique.

As used herein, the term “anti-PEG” refers to any species of antibodies that exhibit specific affinity for a PEG or a PEG-conjugated therapeutic agent. Because there may be more than one epitope on a PEG or a PEG-conjugated therapeutic agent, there may be a range of antibodies that exhibit specific affinities of varying degrees.

For the purpose of this invention, the term anti-PEG is used to broadly refer to all antibodies that exhibit specific affinity for PEGs or PEG-conjugates, and except where indicated otherwise, is not intended to distinguish among different species or subtypes of anti-PEG antibodies. Specificity of an anti-PEG antibody can be easily determined by standard methods known in the art. For example, comparison of antibody binding to PEG and a suitable control substrate maybe used. Alternatively competitive binding assays employing another known anti-PEG may also be suitably used.

As used herein, the term “antigen probe” refers to any substance that is capable of acting as an antigen to an anti-PEG antibody. In some preferred embodiments, the antigen probe is a synthetic agent formed by immobilizing PEGs on a non-cross reacting substrate (i.e. substrates that do not react with anti-PEG). Exemplary non-cross reacting substrates may include red blood cells, glass beads, liposomes, metallic particles, other non-PEG polymeric particles, nitrocellulose, PVDF membrane. The PEG molecules preferably have molecular masses between 300 g/mol to 50,000 g/mol.

Immobilization of PEG is preferably achieved by covalently attaching the PEG molecules to the surface of the substrate. That is, the exposed surface of the substrate such as the surface of the glass beads will become coated by the PEG. Antigen probes formed by covalently attaching PEGs to a non-cross reacting particle are also referred to herein as PEG-coated particles. The density of PEG coating can vary depending on the substrate and the size and homogeneity of the PEG molecules.

When antigen probes are formed by covalently attaching PEG molecules to red blood cells, they are also referred to herein as PEG-RBC. Insertion of a PEG into a red blood cell membrane can be achieved, for example, by using a block copolymer of PEG wherein PEG is covalently attached to a lipophilic block. Some examples of such constructs are PEG-phosholipid, PEG-polyethylene, PEG-lipid, PEG-poly(propylene glycol) block copolymer. Incubation of a PEG-lipophilic block copolymer with a red blood cell rapidly achieves stable insertion of the copolymer into the RBC membrane with the PEG chain extending into the suspension phase. The PEG used in PEG-RBC preferably have molecular masses ranging from 300 g/mol to 50,000 g/mol. In one preferred embodiment, PEGs having molecular mass of about 20,000 g/mol are used.

In addition to PEG-RBC and PEG-coated particles, various PEG polymers in particle form may also be used as antigen probes. These PEG polymers are refereed to herein as PEG particles. Exemplary PEG particles may include the PEG-particles commercially available under the tradename TentaGel-OH particles (Rapp Polymere GmbH, Tübingen, Germany) which are primarily composed of PEG, and are available in discrete sizes from 4 to 300 μm diameter. Preferably, the PEG particles have a diameter ranging from about 2 microns to about 300 microns.

Additionally, other types of particles can be used such as PEG-liposomes, or beads with PEG grafted onto the surface, such as can be prepared by incubation of a chemically reactive PEG-derivative with suitably functionalized polystyrene beads (e.g., amine-functionalized beads).

While the above description of antigen probes envisions the probes as free standing particles that may be suspended or mixed in a solution, this is not a requirement. A person skilled in the art will readily recognize that other forms of presenting the probe may also be used. For example, PEGs can be directly immobilized to a plastic substrate such as in a Coming 96-well plate typically used in the art. In such embodiments, the assay is in a plate format and is easily adaptable for high-throughput screening purposes.

For the purpose of the present invention, the act of contacting the biological samples with the antigen probes may be performed in a number of ways depending on the specific format of the assay, so long as the biological samples come into physical contact with the antigen probes. In those embodiments where the antigen probes are free standing particles, contacting also preferably includes mixing.

It will be appreciated that because the purpose of contacting the biological samples with the antigen probes is to allow the antigen probes to form detectable complexes with any anti-PEG antibodies that may be present in the biological sample, conditions favorable for formation of antigen-antibody complexes should be maintained. One should take care to control any environment factors that may affect the formation of antigen-antibody complexes. Exemplary factors may include temperature, pH, buffer solution, incubation time, etc. PEG and other molecules that contain poly(ethylene glycol) as part of their structure, such as ethoxylated detergents, interfere with binding and should not be used for blocking or as a component of any of the assay solutions. Optimal conditions that will favor complex formation will depend on the specific antigen probe-biological sample pair. Such optimized conditions may be readily determined by any one skilled in the art through routine assay optimization experiments.

Depending on the specific antigen probe-biological sample pair, suitable analysis known in the art may be selected. Exemplary techniques for detecting the antigen-antibody complex in a sample may include fluorescent assay, enzyme-linked assay, flow cytometry, lateral flow assay, or any other techniques commonly known in the art.

In a preferred embodiment, testing for anti-PEG is performed using a simple modification of a routine blood banking serological technique called the immediate spin test, in which the presence of an antigen-antibody reaction is observed as agglutination.

The antigen for this test is poly(ethylene glycol) (PEG) immobilized to a particle surface, preferably a red blood cell (PEG-RBC). The PEG-RBCs are incubated with the sera to be tested for a few minutes and then centrifuged to form a pellet. Examination of the pellet reveals agglutination if the serum contains anti-PEG; no agglutination is observed in the absence of anti-PEG. Typically, non-PEG-coated RBCs from the same source are used as a negative control for confirmation of the result.

In some other preferred embodiments, the antigen probe is a TentaGel-OH particle and the analysis technique is flow cytometry.

Assays according to the above aspect of the present invention will have the advantages that they may be performed with standard biochemical analysis equipment commonly found in analytic labs. The use of the antigen probe to form antigen-antibody antibody complex will ensure that the results of the assays are highly specific for anti-PEG antibodies. These and other characteristics make assays of the present invention uniquely suited for applications in a clinical setting.

Accordingly, in another aspect, the present invention also provides a method for screening a patient prior to administering a PEG-conjugated therapeutic agent. Methods in accordance with this aspect of the present invention will generally have the steps of: (1) obtaining a biological sample from the patient; (2) contacting the biological sample with an antigen probe capable of forming an antigen-antibody complex with an anti-PEG antibody under conditions favorable for formation of the antigen-antibody complex; and (3) analyzing the antigen probe, after having performed step (2), to detect for the presence of the antigen-antibody complex, wherein if the antigen-antibody complex is detected, the presence of anti-PEG in the patient's sample is confirmed, and therefore the patient may be expected to show a diminished response, or no response at all, to the PEG-conjugated therapeutic agent.

Methods according to this aspect of the present invention may be applied on site to biological samples obtained directly from the patients or, alternatively, they may be applied to archived samples for retrospective analysis.

In some patients, exposure to PEG-conjugated therapeutics will elicit an immune response over time. When a patient acquires such an immune response, previously effective PEG-conjugated therapeutics may become less effective or completely ineffective. Therefore, it is important to monitor a patient during the course of treatment to have an accurate and timely assessment of the patient's anti-PEG status. Monitoring protocols may require performing screening assays as previously described above at predetermined time intervals. Such dynamic information can be invaluable to physicians for making clinical decisions about dosage and treatment strategies.

Therefore, in yet another aspect, the present invention also provides a method for monitoring a patient's response to a PEG-conjugated therapeutic agent. Methods in accordance with this aspect of the present invention will generally have the steps of: (1) administering a PEG-conjugated therapeutic agent to a patient; (2) obtaining a biological sample from the patient; (3) contacting the biological sample with an antigen probe capable of forming an antigen-antibody complex with an anti-PEG antibody under conditions favorable for formation of the antigen-antibody complex; (4) analyzing the antigen probe after step (2) to detect for the presence of the antigen-antibody complex; and (5) repeating steps (2)-(4) at predetermined intervals, wherein when the antigen-antibody complex is detected, the presence of anti-PEG is confirmed, indicating that the patient has developed antibodies to PEG and may be expected to show a diminished response, or no response, to further doses of the PEG-conjugated therapeutic agent.

In still another aspect of the present invention, there is provided a kit for performing assays and methods as described in the above aspects of the present invention. In generally, design of kits in accordance with embodiments of the present invention will have the object of providing standardized reagents, convenience of storage, transportation, and ease of operation.

In a preferred embodiment, the core component of a kit according to embodiments of the present invention is an antigen probe capable of forming an antigen-antibody complex with an anti-PEG antibody. Antigen probes may be provided in the kit in various forms, including dry power form, solutions, pre-suspended particles, or other formulations commonly known in the art. In some embodiments, one or more control sample(s) may also be included to facilitate comparison and quality control of the assay result. Control samples may be either positive control in which the samples are known to be anti-PEG positive, or negative control samples in which the samples are known to be anti-PEG negative. A combination of positive and negative controls may also be included.

To enhance the utility and user convenience, other components may also be included in the kit. For example, in some embodiments, the kit may include apparatus such as needle and test tube combinations for obtaining biological samples; pre-measured reagent portions; and inserts for protocols and other critical information. The kit may further include a PEG-conjugated therapeutic agent along with a set of sampling handling tools (e.g. syringes, test tubes, surgical gloves, etc.) for administering and monitoring the PEG-conjugated therapeutics agent.

Having generally described this invention, a further understanding can be obtained by reference to the following specific examples and the accompanying figures. It will be understood by those skilled in the art that these specific examples and figures represent only exemplary embodiments of the present invention. It will be appreciated that the embodiments disclosed herein are for illustrative purposes only and are not to be construed as limiting in any way. Various modifications and alterations might be made by those skilled in the art without departing from the spirit and scope of the invention as set forth in the attached claims.

### EXAMPLES

### Example 1

### Testing For An Antibody To Poly(Ethylene Glycol):

### Method:

### Preparation of Antigen:

Blood type O red blood cells (RBCs) were washed 3 times with phosphate buffered saline (PBS, pH 7.4, 290 mOsm/kg) at 1400×g for 6 minutes. RBCs were then resuspended to a 10% hematocrit (hct) in 15 mM triethanolamine buffer (pH 8.4, 290 mOsm/kg). Poly(ethylene glycol) coating of RBCs was achieved by the addition of a reactive PEG to the RBC suspension. A succinimidyl propionate derivate of monomethoxy-poly(ethylene glycol) of molecular mass 20 kDa (mPEG20 k-SPA) was dissolved in cold 10 mM hydrochloric acid +154 mM NaCl, and added to the RBC suspension to yield a suspension phase concentration of 5 mg/mL mPEG20 k-SPA. The mixture was incubated at room temperature for 1 hour, and then washed 3 times with PBS at 500×g for 10 minutes. PEG-RBCs were then resuspended to a 5% hct in PBS and used for serologic testing.

### Tube Test for Anti-PEG:

One drop of RBCs (PEG-coated or control (uncoated) RBCs) at 5% hct were added to 2 drops of plasma. Samples were incubated at room temperature for 15 minutes and then centrifuged at 1000×g for 1 minute. Agglutination of PEG-coated or uncoated (control) RBCs by each plasma sample was scored according to the 0-4+ scale. An anti-PEG positive sample was identified as one where agglutination of PEG-RBCs was observed in the absence of agglutination of control (uncoated) RBCs. An anti-PEG negative sample was identified as one where no agglutination was observed with PEG-RBCs.

### Confirmation of Specificity of Anti-PEG:

To confirm specificity of anti-PEG following a positive serological test with PEG-RBCs, the test was repeated following removal of anti-PEG from the sample by pre-incubation with PEG-particles: Four hundred μL of anti-PEG positive plasma were added to 20 mg of 10 μm TentaGel-OH suspended in 200 L of PBS and incubated at room temperature for 30 minutes to specifically adsorb any anti-PEG present. The mixture was then centrifuged at 1000×g for 5 minutes, and the supernatant separated for testing. Testing was repeated as described above with PEG-RBCs. Anti-PEG specificity was confirmed if the agglutination of PEG-RBCs was eliminated after adsorption with PEG.

### Results:

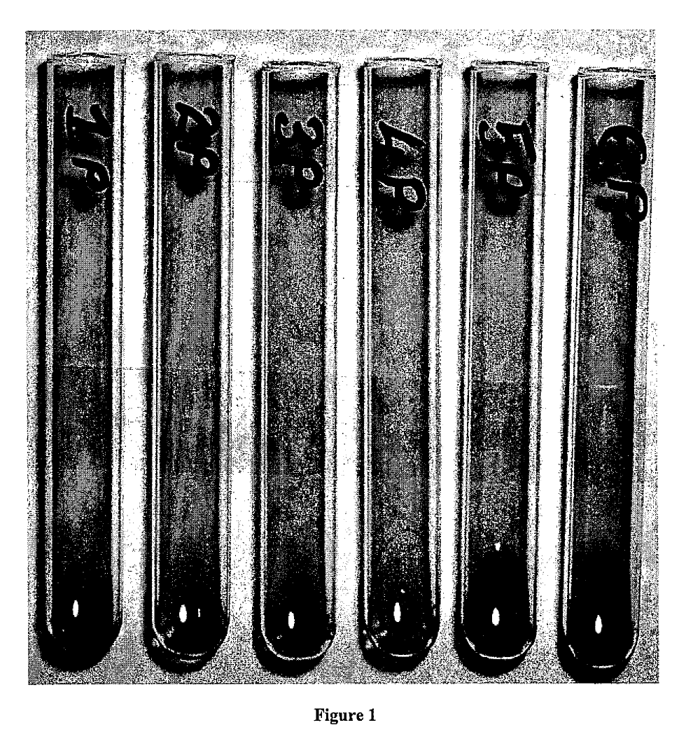

The results of serological testing with PEG-RBCs are shown in FIG. 1. No agglutination was observed with uncoated (control) RBCs (images not shown). The sample details for FIG. 1 are as follows: Sample 1P) shows PEG-RBCs in buffer (negative); Samples that are anti-PEG positive are 2P) weak positive (2+w); 3P) weak positive (1+−2+w) and 4P) strong positive (4+). The anti-PEG specificity was confirmed by adsorption of sample 4P with PEG-particles, resulting in an absence of agglutination (sample 5P). Sample 6P tested negative for anti-PEG (i.e., no agglutination of PEG-RBCs).

### Example 2

### Gel Test For An Antibody To Poly(Ethylene Glycol)

### Preparation of Antigen:

PEG-coated RBCs were prepared as described in Example 1.

Preparation of Gel Test tubes:

Gel Test tubes were prepared as follows. One hundred microliters of Sephaeryl 500-HR beads at 50% solids were pipetted into a narrow 300 μL tube. The tube was centrifuged at 500×g for 10 minutes.

### Example of Gel Test for Anti-PEG:

One hundred microliters of plasma was then pipetted on the top of the gel layer. Twenty five microliters of PEG-RBCs (or control (uncoated) RBCs) at 10% hct were added to the top of the plasma layer. Samples were incubated at room temperature for 15 minutes, and then centrifuged at 500×g for 3 minutes

Agglutination was scored accordingly: RBCs do not enter top of gel =4+ positive test, RBCs pass through gel=0 negative test.

### Results:

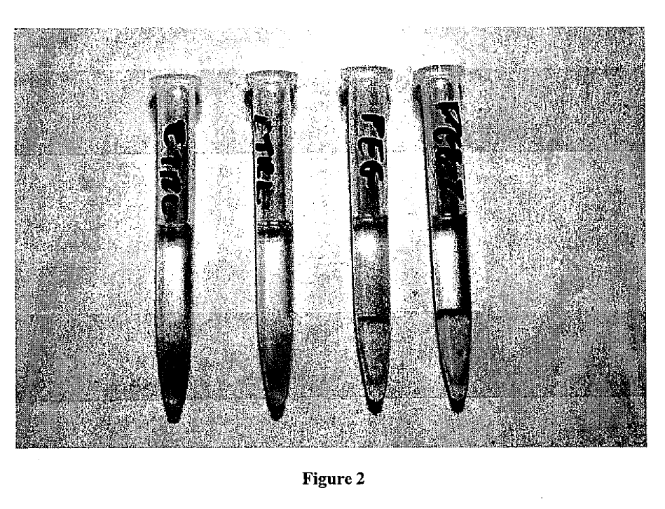

The results of the gel test with PEG-RBCs are shown in FIG. 2. All uncoated (control) RBCs passed through the gel and gave a negative gel test (images not shown). The sample details for FIG. 2 are as follows: Plasma samples labeled “Ctrl” are anti-PEG negative samples as PEG-RBCs passed through the gel giving a negative gel test. Plasma samples labeled “PEG” and “PEG2” are anti-PEG positive showing a strong 4+ agglutination test result.

### Example 3

### Flow Cytometric Testing For An Antibody To Poly(Ethylene Glycol) Method:

Fifty microliters of each test plasma were added to 100 μL of PBS and 25 μL of a 1% suspension of 10 μm diameter poly(ethylene glycol) particles, known commercially as TentaGel-OH beads (TentaGel-OH M 30 100, Rapp Polymere GmbH, Tübingen, Germany), which are composed primarily of PEG. The mixture was incubated for 1 hour at room temperature and the beads were washed twice with PBS (200×g for 2 minutes) and resuspended with 1 mL of PBS containing 5 μL of fluorescein isothiocyanate labeled-anti-human IgG and 5 μL of R-phycoerythrin labeled-anti-human IgM. After 1 hour incubation at room temperature in the dark, the particles were washed 3 times with PBS (200×g for 2 minutes) and resuspended with 0.5 mL of PBS and examined by flow cytometry. Ten thousand counts were recorded per sample, gated for single beads. Non-specific protein uptake was investigated by staining with FITC-anti-human albumin.

### Results:

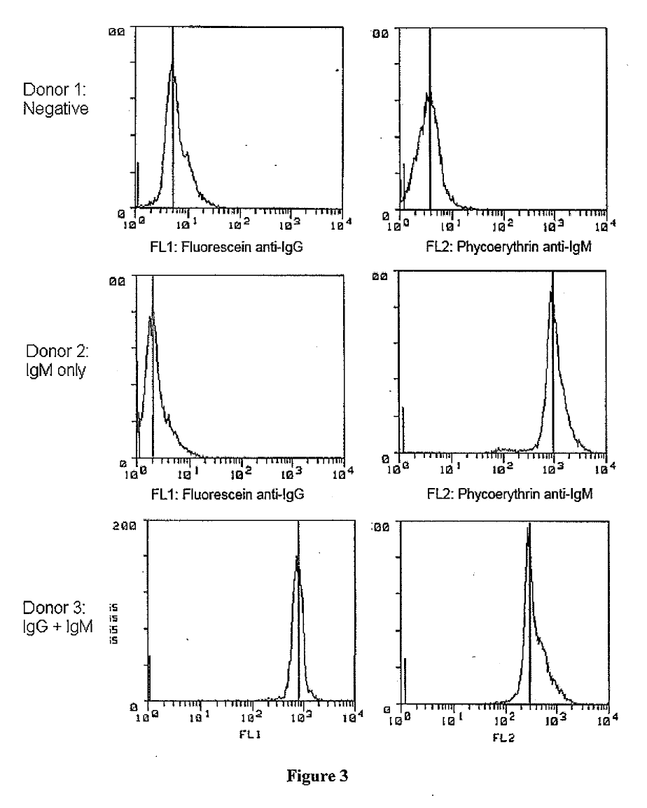

Representative data for human plasma samples are shown in FIG. 3. Donor 1 shows no evidence of bound antibody (anti-PEG negative), Donor 2 shows evidence of IgM binding only (anti-PEG positive, IgM), Donor 3 shows binding of both IgG and

IgM (anti-PEG positive, IgG and IgM). No evidence of albumin binding to TentaGel-OH beads was observed for all sera tested.

### Example 4

### Variation of Reactive PEG Concentration:

### Method

Freshly drawn blood type O RBCs were PEG-coated according to the protocol described in Example 1, but with the following variations to demonstrate the effect of incubation hematocrit (hct) and reactive PEG concentration:
Incubation at 50% hct:

Washed RBCs were resuspended in triethanolamine buffer at 50% hct. A succinimidyl propionate derivate of monomethoxy-poly(ethylene glycol) of molecular mass 20 kDa (mPEG20k-SPA) was dissolved in cold 10 mM hydrochloric acid+154 mM NaCl and added to RBC alquots to achieve the following concentrations:
1) 5 mg/mL of suspension (10 mg/mL RBCs)
2) 10 mg/mL of suspension (20 mg/mL RBCs)
3) 20 mg/mL of suspension (40 mg/mL RBCs)

Washed RBCs were resuspended in triethanolamine buffer at 10% hct. A succinimidyl propionate derivate of monomethoxy-poly(ethylene glycol) of molecular mass 20 kDa (mPEG20k-SPA) was dissolved in cold 10 mM hydrochloric acid+154 mM NaCl and added to RBC alquots to achieve the following concentrations:
1) 1 mg/mL of suspension
2) 5 mg/mL of suspension
3) 10 mg/mL of suspension

The mixtures were incubated at room temperature for 1 hour, and then washed 3 times with PBS at 500×g for 10 minutes. PEG-RBCs were then resuspended to a 5% hct in PBS and used for serologic testing. PEG-coated RBCs were evaluated with autologous plasma (Sample 1, anti-PEG negative) and two anti-PEG positive plasma samples (Samples 2 and 3) using tube and gel tests described in Example 1 and Example 2 respectively.

### Results:

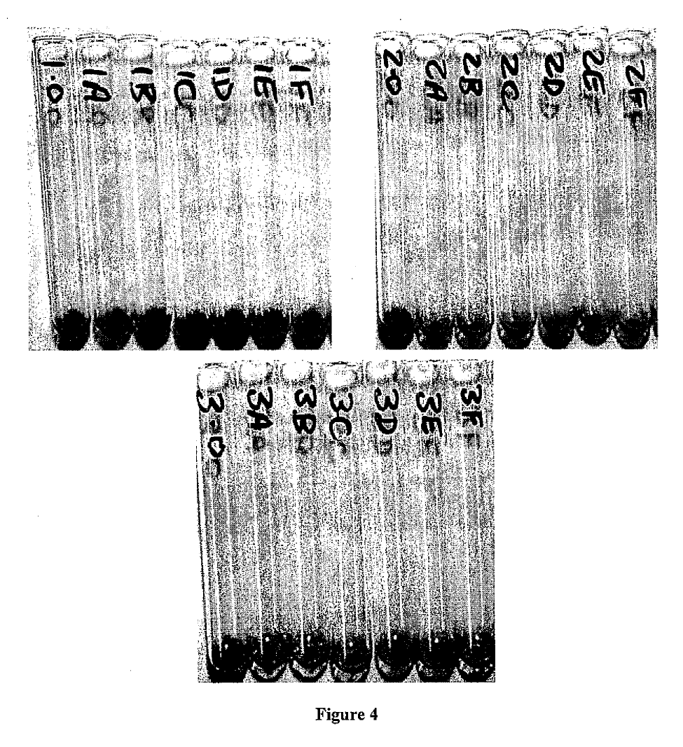

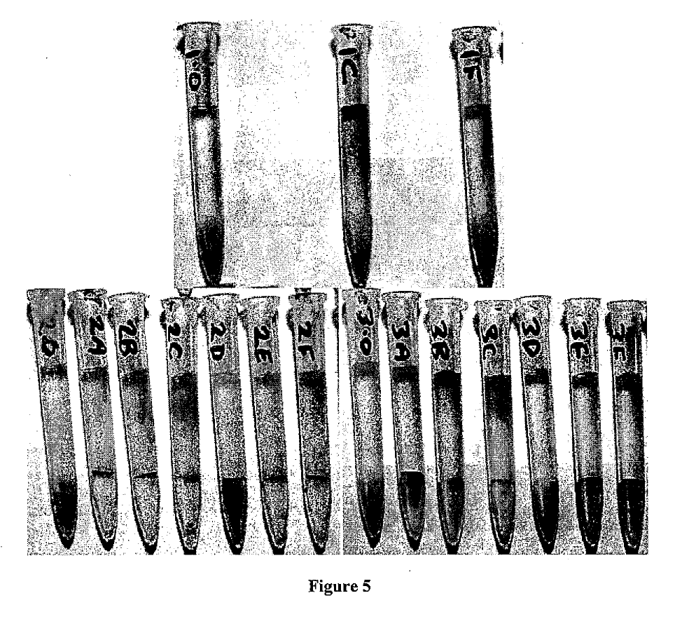

Table 1 below shows the tube and gel test results for PEG-RBCs (and control, uncoated RBCs) incubated with three plasma samples (Sample 1=negative for anti-PEG, Samples 2 and 3=positive for anti-PEG). Images of the tube and gel test samples are also shown in FIGS. 4 and 5.

TABLE 1
PEG-coating details, tube and gel test results for anti-PEG
with 3 plasma samples (Sample 1 = negative for anti-PEG,
Samples 2 and 3 = positive for anti-PEG).

| | Sample Label | | | | | | |
|---|---|---|---|---|---|---|---|
| | 0 | | | | | | |
| Details Label | (Ctrl) | A | B | C | D | E | F |
| Incubation hct | N/A | 50% | | | 10% | | |
| Reactive PEG | 0 | 5 | 10 | 20 | 1 | 5 | 10 |
| Conc (mg/mL) | | | | | | | |

Tube Test

| | | | | | | | |
|---|---|---|---|---|---|---|---|
| Sample 1 (Neg) | 0 | 0 | 0 | 0 | 0 | 0 | 0 |
| Sample 2 (pos) | 0 | 4+ | 4+ | 4+ | 2+/3+ | 4+ | 4+ |
| Sample 3 (pos) | 0 | 3+ | 3+ | 3+ | 2 + w | 3+ | 4+ |

Gel Test

| | | | | | | | |
|---|---|---|---|---|---|---|---|
| Sample 1 (neg) | 0 | | | 0 | | | 0 |
| Sample 2 (pos) | 0 | 4+ | 4+ | 4+ | 1+ | 4+ | 4+ |
| Sample 3 (pos) | 0 | 2+ | 4+ | 4+ | 0 | 3+ | 4+ |

### Conclusions:

Preparation of reagent PEG-RBCs for screening plasma samples for the presence of anti-PEG may be performed using an incubation hematocrit of 10-50% and a reactive PEG concentration of 5 to 10 mg/mL at 10% hot, and 10 to 20 mg/mL at 50% hot.

### Example 5

### Identification of Anti-PEG Epitope

PEG-RBCs were prepared and serological testing performed as described in Example 1. Three anti-PEG positive sera that gave a strong agglutination (4+) result with PEG-RBCs using the tube test were used for the anti-PEG epitope study. Two percent (w/v) solutions of various polymers [PEG of molecular mass 300 g/mol and 20,000 g/mol, monomethoxy-PEG of molecular mass 5000 g/mol, dextran of molecular mass 40,000 g/mol, polyvinylalcohol (PvOH) of molecular mass 25,000 g/mol, polypropylene glycol) (PPG) of molecular mass 2000 g/mol] and small ethers and ether oligomers [di- to penta-(ethylene glycol); di- to tetra-(ethylene glycol) dimethyl ether] were prepared in PBS. The polymer and small ether solutions were added to anti-PEG positive sera at a 1:1 (v/v) ratio giving a final polymer/small ether concentration of 1% (w/v) and incubated for 30 minutes at room temperature. Agglutination testing with PEG-RBCs was then performed as described in Example 1. Inhibition of agglutination by the smallest ether molecule tested determined the anti-PEG epitope.

### Results:

Complete inhibition of agglutination (from 4+ to 0) was observed in the presence of all PEGs (MW 300-20,000 g/mol), PPG (2000 g/mol), tri- and tetra-(ethylene glycol)dimethyl ether and penta(ethylene glycol). Di(ethylene glycoldiethyl ether and tetra(ethylene glycol) reduced PEG-RBC agglutination (4+ to 2+). Dextran, PvOH, ethylene glycol, di- and tri-(ethylene glycol) had no effect.

### Conclusion:

Comparison of the smallest inhibitors indicates that the minimum epitope required for binding of the PEG-antibody is a backbone of 4 to 5 repeat —(C—O—C)— units.

TABLE 2
Agglutination Of PEG-RBCs by 3 normal plasma samples (4+ reactive for anti-PEG) in presence Of 1%
w/v PEG, PEG Oligomers and analogs, PPG, and irrelevant polymers (dextran 40 and polyvinyl alcohol).

| | Agglutination Score | | | |
|---|---|---|---|---|
| Polymer/Compound added | Anti-PEG Positive Donor | | | Ether/Oligomer/Polymer |
| (all at 1% w/v) | A | B | C | Backbone Structure |
| Ethlyene Glycol | 3+ | 4+ | 4+ | O—CC—O |
| Diethylene Glycol | 3+ | 4+ | 4+ | O—CC—O—CC—O |
| Triethylene Glycol | 2+ | 4+ | 3+ | O—CC—O—CC—O—CC—O |
| Diethylene Glycol Dimethyl Ether | 0+ | 2+ | 2+ | C—O—CC—O—CC—O—C |
| Tetraethylene Glycol | 7+ | 2+ | 1+ | O—CC—O—CC—O—CC—O—CC—O |
| Diethylene Glycol Diethyl Ether | 0+ | 1+ | 1+ | CC—O—CC—O—CC—O—CC |
| Pentaethylene Glycol | 1+ | 1+ | 0+ | O—CC—O—CC—O—CC—O—CC—O—CC—O |
| Triethylene Glycol Dimethyl Ether | 0 | 0 | 0 | C—O—CC—O—CC—O—CC—O—C |
| Tetraethylene Glycol Dimethyl Ether | 0 | 0 | 0 | C—O—CC—O—CC—O—CC—O—CC—O—C |
| PEG 300 | 0 | 0 | 0 | O—CC—O—CC—O—CC—O—CC—O—CC—O—CC—O |
| mPEG 5000 | 0 | 0 | 0 | C—O—CC—O—(CC—O)₁₁₀—CC—O—CC—O—CC —O |
| PEG 20,000 | 0 | 0 | 0 | O—CC—(O—CC)₄₅₀—O—CC—O—CC—O—CC —O |
| PPG 2000 | 0 | 0 | 0 | O—CC—(O—CC)₃₁O—CC—O—CC—O—CC —O |
| Dextran 40,000 | 3+ | 4+ | 4+ | |
| PvOH 25,000 | 3+ | 4+ | 4+ | |
| PBS (control) | 4+ | 4+ | 4+ | |

### Example 6

### Serology Screening Of Healthy Blood Donors For Anti-PEG

Plasma samples were collected from 350 normal healthy subjects. One drop of RBCs (PEG-coated or control (uncoated) RBCs) at 5% hct were added to 2 drops of plasma. Samples were incubated at room temperature for 15 minutes and then centrifuged at 500×g for 1 minute. Agglutination of PEG-coated or uncoated (control) RBCs by each plasma sample was determined using the serological tube test, and agglutination scored according to the 0-4+ scale.

### Results:

94 plasma samples (26.9%) agglutinated PEG-RBCs (26.9% positive for anti-PEG), 74 of these (21.2%) scored 1+ to 2+, and 20 (5.7%) showed strong agglutination (3+ or 4+) (Table 3). No agglutination was observed for control (uncoated) RBCs in patient sera.

TABLE 3
Testing of normal donor sera with PEG-coated
RBCs scored according to the 0-4+ scale.

| Agglutination score | Number | Percentage |
|---|---|---|
| 0 | 256 | 73.1 |
| 1+ | 31 | 8.9 |
| 2 + w | 26 | 7.4 |
| 2+ | 17 | 4.9 |
| 3+ | 12 | 3.4 |
| 4+ | 8 | 2.3 |
| Negative | 256 | 73.1 |
| Positive | 94 | 26.9 |
| Total | 350 | 100 |

### Example 7

### Flow Cytometric Screening Of Healthy Blood Donors For Anti-PEG and Identification of IgG Sub-Types

### Method

Fifty microliters of test plasma were added to 100 μL of PBS and 25 μL of a 1% suspension of 10 μm diameter poly(ethylene glycol) particles, known commercially as TentaGel-OH beads (TentaGel-OH M 30 100, Rapp Polymere GmbH, Tübingen, Germany), which are composed primarily of PEG. The mixture was incubated for 1 hour at room temperature and the beads were washed, twice with PBS (200×g for 2 minutes) and resuspended with 1 mL of PBS containing 5 μL of fluorescein isothiocyanate labeled-anti-human IgG and 5 μL of R-phycoerythrin labeled-anti-human IgM. After 1 hour incubation at room temperature in the dark, the particles were washed 3 times with PBS (200×g for 2 minutes) and resuspended with 0.5 mL of PBS and examined by flow cytometry. Ten thousand counts were recorded per sample, gated for single beads.

### Testing for IgG Sub-Types:

Twelve anti-PEG positive sera, and one anti-PEG negative serum were also examined for IgG sub-types. Sera were incubated with 10 μm diameter TentaGel-OH beads as described above. Each sample was divided into 4 equal aliquots and stained for bound IgG sub-types with 1 mL of PBS containing 5 μL of fluorescein isothiocyanate labeled-anti-human IgG-1, IgG-2, IgG-3 or IgG-4. Flow cytometric analyses were performed as described above.

### Results:

Flow cytometric analysis of TentaGel-OH beads showed 97 samples (27.7%) positive for IgG and/or IgM (27.7% positive for anti-PEG), of which 67 samples (19.1%) showed IgG binding only, 18 (5.1%) showed IgM only, and 12 (3.4%) showed both IgG and IgM uptake (Table 4). No evidence of albumin uptake was observed, which argues against non-specific protein binding. Analysis of 12 anti-PEG positive sera for IgG subtypes showed that of 11 sera that were positive of anti-PEG IgG, all 11 were positive for anti-PEG IgG-2, and one sera sample was positive for IgG-1, IgG-2 and IgG-3 (Table 5). One anti-PEG positive sera that was anti-PEG IgM only (sample labeled “V” in Table 5) tested negative for all IgG subtypes analyzed No anti-PEG positive sample showed evidence of IgG-4.

TABLE 4
Flow cytometric analysis of TentaGel-OH beads incubated
in normal donor sera and stained for bound IgG and IgM.
Mean fluorescence data were employed to identify the
presence or absence of anti-PEG in test sera.

| | Number | Percentage |
|---|---|---|
| IgG only | 67 | 19.1 |
| IgM only | 18 | 5.1 |
| IgG + IgM | 12 | 3.4 |
| Negative | 253 | 72.3 |
| Positive | 97 | 27.7 |
| Total | 350 | 100 |

TABLE 5
Flow cytometric analysis of TentaGel-OH beads incubated in normal
donor anti-PEG positive sera and stained for bound IgG sub-types
(IgG-1, -2, -3 and -4). Mean fluorescence data for each analysis
is given, with a positive result given in bold text.

| | Mean Fluorescence Intensity Of TentaGel-OH | | | | |
|---|---|---|---|---|---|
| | beads | | | | Anti-PEG |
| Donor # | IgG-1 | IgG-2 | IgG-3 | IgG-4 | Sub-Type |
| P | 7.3 | 4.9 | 17 | 0.6 | IgG-1, -2 |
| | | | | | and -3 |
| T | 0.4 | 6.1 | 0.4 | 0.4 | IgG-2 |
| 219 | 0.4 | 1.5 | 0.5 | 0.4 | IgG-2 |
| 238 | 0.5 | 1.4 | 0.5 | 0.4 | IgG-2 |
| 243 | 0.5 | 1.1 | 0.4 | 0.3 | IgG-2 |
| 253 | 0.4 | 2 | 0.3 | 0.3 | IgG-2 |
| 267 | 0.6 | 11.4 | 0.5 | 0.4 | IgG-2 |
| 282 | 0.6 | 1.7 | 0.5 | 0.4 | IgG-2 |
| 292 | 0.5 | 4.6 | 0.6 | 0.3 | IgG-2 |
| 294 | 0.6 | 2.1 | 0.6 | 0.5 | IgG-2 |
| 299 | 0.4 | 5.8 | 0.4 | 0.3 | IgG-2 |
| V | 0.3 | 0.3 | 0.4 | 0.3 | (IgM Only) |
| J | 0.3 | 0.2 | 0.4 | 0.3 | Negative |
| | | | | | Control |

### Example 8

### Testing Of Patient Sera For Anti-PEG

### Methods:

Stored serum samples were collected from 28 pediatric acute lymphoblastic leukemia (ALL) patients who received the chemotherapy agent, PEG-conjugated asparaginase (PEG-ASNase). Sera were assayed for ASNase activity using a microplate technique. Testing for anti-PEG was performed by serology as described in Example 1 and by flow cytometry as described in Example 3.

### Results:

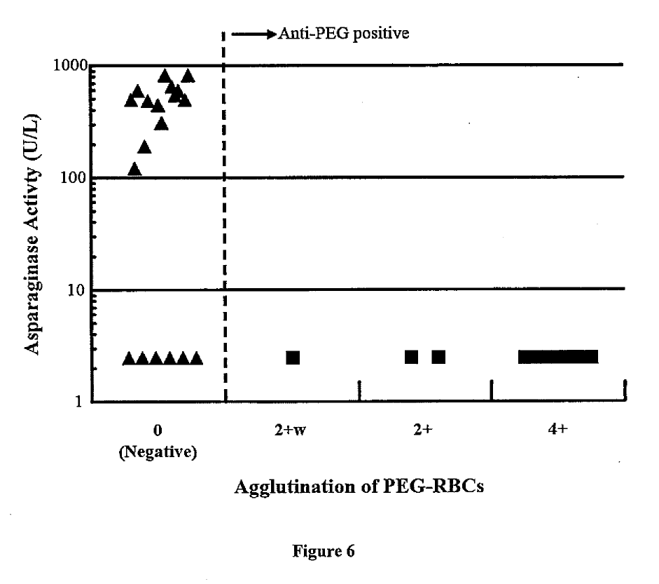

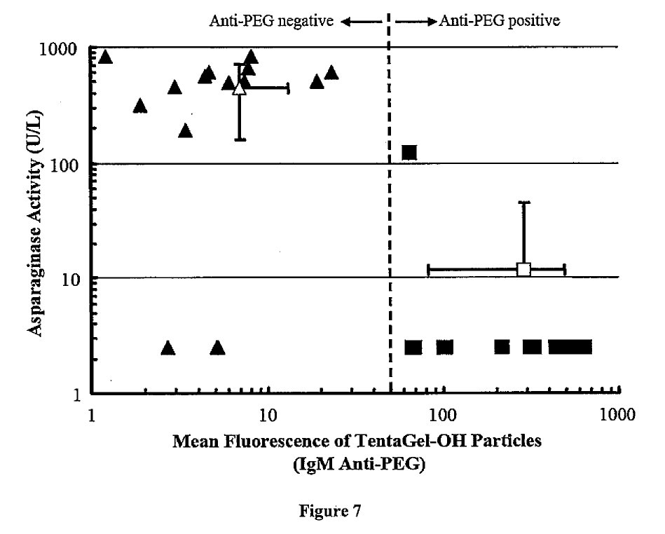

Regardless of which technique was used to determine anti-PEG (i.e., serology or flow cytometry), all PEG-ASNase-treated patient sera that were anti-PEG positive showed low or undetectable ASNase activity (FIGS. 6 and 7). Only one anomalous result was observed: one sample was weakly positive for anti-PEG IgM (mean fluorescence of 65) and also positive for anti-ASNase, but had ASNase activity at the low end of the therapeutic range (123 U/L).

### Conclusion:

The presence of anti-PEG is very closely associated with rapid clearance of PEG-ASNase for the samples analyzed in this study.

### REFERENCES CITED

(1) Richter A W, Åkerblom E. Antibodies against polyethylene glycol produced in animals by immunization with monomethoxy polyethylene glycol modified proteins. Int Arch Allergy Appl Immunol. 1983;70:124-131.
(2) Richter A W, Åkerblom E. Polyethylene glycol reactive antibodies in man: titer distribution in allergic patients treated with monomethoxy polyethylene glycol modified allergens or placebo, and in healthy blood donors. Int Arch Allergy Appl Immunol. 1984;74:36-39.
(3) Ganson, N J, Kelly S J, Scarlett E, Sundy J S, Hershfield, M S. Control of hyperuricemia in subjects with refractory gout, and induction of an antibody against poly(ethylene glycol) (PEG), in a phase I trial of subcutaneous PEGylated urate oxidase. Arthritis Res Ther. 2006;8:R12.

What is claimed is:

1. A method for detecting the presence of anti-poly(ethylene glycol) antibodies in a biological sample, comprising:
(1) providing an antigen probe capable of forming an antigen-antibody complex with anti-poly(ethylene glycol) antibodies;
(2) contacting said antigen probe with said biological sample under conditions favorable for formation of an antigen-antibody complex;
(3) analyzing said antigen probe to detect the presence of said antigen-antibody complex;
wherein the presence of said antigen-antibody complex indicates that anti-poly(ethylene glycol) antibodies were present in said biological sample.

2. The method of claim 1, wherein the analyzing step further comprises estimating the concentration of anti-poly(ethylene glycol) antibodies in a biological sample

3. The method of claim 1 wherein an enzyme-conjugated antibody is used to detect or quantitate the antibody-antigen complex.

4. The method of claim 1 wherein a particle agglutination technique is employed to detect or quantitate the antibody-antigen complex.

5. The method of claim 1 wherein a fluorochrome-conjugated antibody is used to detect or quantitate the antibody:antigen complex.

6. The method of claim 1 wherein the biological fluid is blood, serum or plasma.

7. The method of claim 1 wherein the antigen probe comprises an inert solid substrate upon which polymer molecules have been immobilized; said polymer molecules containing a sequence of at least 5 consecutive alkylene oxide repeat units, wherein said sequence represents the antigen required to form an antigen-antibody complex with an anti-poly(ethylene glycol) antibody.

8. The method of claim 7 wherein the solid substrate comprises poly(ethylene-glycol)-coated blood cells.

9. The method of claim 7 wherein the solid substrate comprises poly(ethylene-glycol)-coated liposomes.

10. The method of claim 7 wherein the solid substrate comprises inert particles with a diameter less than one millimeter.

11. The method of claim 7 wherein the inert particles have a diameter of about 10 microns.

12. The method of claim 7 wherein the insert particles are comprised essentially of poly(ethylene glycol).

13. The method of claim 12 wherein said inert particles are TentaGel particles.

14. The method of claim 7 wherein the polymer molecules immobilized on the solid substrate are poly(ethylene glycol) molecules with a molecular mass from about 300 g/mol to about 50,000 g/mol.

15. The method of claim 7 wherein the polymer molecules immobilized on the solid substrate are poly(ethylene glycol) molecules with a molecular mass of about 20,000 g/mol.

16. The method of claim 7 wherein the polymer molecules are covalently bonded to the solid substrate.

17. The method of claim 7 wherein the solid substrate comprises red blood cells to which poly(ethylene glycol) molecules have been covalently bonded.

18. The method of claim 7 wherein the sequence of at least 5 consecutive alkylene oxide repeat units is provided by poly(ethylene glycol) blocks in block copolymer.

19. The method of claim 7 wherein the polymer molecules immobilized on the solid substrate are methoxy-poly(ethylene glycol) molecules.

20. A method for screening a patient prior to administering a poly(ethylene glycol)-conjugated therapeutic agent, comprising:
(1) obtaining a biological sample from the patient;
(2) contacting the sample with an antigen probe capable of forming an antigen-antibody complex with anti-poly(ethylene glycol) antibodies, under conditions favorable for formation of an antigen-antibody complex; and
(3) analyzing the antigen probe to detect the presence of said antigen-antibody complex;
wherein if said antigen-antibody complex is detected, the presence of an anti-poly(ethylene glycol) antibody in the patient's sample is confirmed, and the patient is predicted to show a diminished response, or no response at all, to the PEG-conjugated therapeutic agent.

21. A method for monitoring a patient's response to a PEG-conjugated therapeutic agent, comprising:
(1) obtaining a biological sample from the patient after administration of the PEG-conjugated therapeutic agent;
(2) contacting the biological sample with an antigen probe capable of forming an antigen-antibody complex with anti-poly(ethylene glycol) antibodies, under conditions favorable for formation of an antigen-antibody complex;
(3) analyzing the antigen probe to detect the presence of said antigen-antibody complex;
(4) repeating steps (1) to (3) at appropriate intervals thereafter, as required;
wherein if the antigen-antibody complex is detected, it indicated that the patient has developed antibodies to poly(ethylene glycol) and may be expected to show a diminished response, or no response at all, to further doses of the poly(ethylene glycol)-conjugated therapeutic agent.

22. A method for predicting and for monitoring a patient's response to a poly(ethylene glycol)-conjugated therapeutic agent, comprising:
(1) obtaining a biological sample from the patient;
(2) contacting the biological sample with an antigen probe capable of forming an antigen-antibody complex with anti-poly(ethylene glycol) antibodies, under conditions favorable for formation of an antigen-antibody complex;
(3) analyzing the antigen probe to measure the amount of anti-poly(ethylene glycol) antibody present in said antigen-antibody complex;
(4) deciding whether to administer the poly(ethylene glycol)-conjugated therapeutic agent, and at what dosage, based upon the results of step (3); and
(5) repeating steps (1) to (4) at appropriate intervals thereafter, until treatment with the poly(ethylene glycol)-conjugated therapeutic agent is completed or discontinued.

23. The method of claim 20 wherein the therapeutic agent is selected from the group consisting of: asparaginase, insulin, adenosine deaminase, arginine deaminase, glutaminase, camptothecin, filgrastin, recombinant human growth hormone, interferon, uricase, anti-tumor necrosis factor, anti-vascular endothelial growth factor, anti-granulocyte-stimulating factor, anti-granulocyte-macrophage colony stimulating factor, anti-glutamic acid decarboxylase, anti-bacillus anthracis exotoxin, photosensitizer immunoconjugates, benzoporphyrin derivative-anti-epidemial growth factor receptor, superoxide dismutase, glucocerebrosidase, hemoglobin, and liposome.

24. The method of claim 20 wherein an enzyme-conjugated antibody is used to detect or quantitate the antibody-antigen complex.

25. The method of claim 20 wherein a particle agglutination technique is employed to detect or quantitate the antibody-antigen complex.

26. The method of claim 20 wherein a fluorochrome-conjugated antibody is used to detect or quantitate the antibody-antigen complex.

27. The method of claim 20 wherein the biological sample is blood, serum or plasma.

28. The method of claim 20 wherein the antigen probe comprises an inert solid substrate upon which polymer molecules have been immobilized; said polymer molecules containing a sequence of at least 5 consecutive alkylene oxide repeat units, wherein said sequence represents the antigen required to form an antigen-antibody complex with an anti-poly(ethylene glycol) antibody.

29. The method of claim 28 wherein the solid substrate comprises poly(ethylene-glycol)-coated blood cells.

30. The method of claim 28 wherein the solid substrate comprises poly(ethylene-glycol)-coated liposomes.

31. The method of claim 28 wherein the solid substrate comprises inert particles with a diameter less than one millimeter.

32. The method of claim 28 wherein the inert particles have a diameter of about 10 microns.

33. The method of claim 28 wherein the insert particles are comprised essentially of poly(ethylene glycol)

34. The method of claim 33, wherein the inert particles are TentaGel particles.

35. The method of claim 28 wherein the polymer molecules immobilized on the solid substrate are poly(ethylene glycol) molecules with a molecular mass from about 300 g/mol to about 50,000 g/mol.

36. The method of claim 28 wherein the polymer molecules immobilized on the solid substrate are poly(ethylene glycol) molecules with a molecular mass of about 20,000 g/mol.

37. The method of claim 28 wherein the polymer molecules are covalently bonded to the solid substrate.

38. The method of claim 28 wherein the solid substrate comprises red blood cells to which poly(ethylene glycol) molecules have been covalently bonded.

39. The method of claim 28 wherein the sequence of at least 5 consecutive alkylene oxide repeat units is provided by poly(ethylene glycol) blocks in block copolymer.

40. The method of claim 28 wherein the polymer molecules immobilized on the solid substrate are methoxy-poly(ethylene glycol) molecules.

41. A kit for performing a screening assay according to claims 1 comprising:
an antigen probe capable of forming an antigen-antibody complex with an anti-poly(ethylene glycol) antibody

42. The kit of claim 41 further comprising one or more control sample(s), wherein the control sample(s) can be positive control sample, negative control sample, or combinations thereof.
